(19) United States
(12) Patent Application Publication
(10) Pub. No.: US 2006/0106330 A1
(43) Pub. Date: May 18, 2006

(54) FACIAL LIFTING AND TONING DEVICE

(76) Inventors: Victor Andrade, Ann Arbor, MI (US); Lorena Andrade, Ann Arbor, MI (US)

Correspondence Address: FOSTER, SWIFT, COLLINS & SMITH, P.C., 313 SOUTH WASHINGTON SQUARE, LANSING, MI, 48933, US

(21) Appl. No.: 10/904,553

(22) Filed: Nov. 16, 2004

(51) Int. Cl. A61F 13/12

(52) U.S. Cl. 602/74

(57) ABSTRACT

A facial toning device that creates a youthful look by toning and firming the facial muscles by applying pressure, adjustable to a user's preference. The facial device is a one-piece stretchable material that wraps around the wearer's head and jaw line. Along its edges there is a strong and comforting facial reinforcement. There are openings for chin and ears, which will allow the wearer to be more comfortable while wearing the facial device. Along the side of the facial device there are two adjustable bands, which allow a wearer to vary the amount of pressure being applied to the face. The optional eye attachment provides for a sleeping aid and/or compartment for hot or cold sources, which may be worn while the wearer is wearing the facial device in their sleep or relaxing.

### FIELD OF THE INVENTION

The present invention relates to a facial lifting and toning device, and particularly to a facial lifting and toning device having a stretchable material, which applies pressure, through adjustable bands, to a wearer's face muscles and skin.

### BACKGROUND OF THE INVENTION

Face-lifts, massages, creams and many other techniques have been performed throughout history to improve and preserve facial beauty. Devices have also been introduced to make a face look younger and more appealing. Masks and the like have been developed to prevent or even rehabilitate aged facial skin.

Young (U.S. Pat. No. 4,694,823) discloses a neck and facial lift band assembly, which is accomplished by a yoke assembly that has a central chin segment connected to a pair of jaw segments, while attached by a “criss-crossed” strap from behind the head. Unfortunately, this device is too complicated with its “criss-crossed” bands in the back and it only supports the neck and bottom portion of the jaw. In short, it does not provide much facial support.

Nash-Morgan (U.S. Pat. No. 5,116,675) discloses a device having an adhesive neck and facial wrinkle-gathering feature to be worn in short periods. This patent seems to be a burden on the wearer since it can only be worn for a short period of time and it uses adhesives that attach directly to the skin and would be uncomfortable to remove.

Kelly et al. (U.S. Pat. No. 6,039,710) discloses a facial apparatus that provides facial support with a head band, cheek support, head strap, nose strap, and forehead strap. The cheek support and head strap attach to two locations of the head band. This device also has a nose support to define the aperture for the nose. While this device attempts to reduce facial wrinkling it is a very complicated and burdensome for the wearer.

Klein (U.S. Pat. No. 5,396,881) discloses a facial mask pressed against a wearer's facial muscles by pressurizing an expansible chamber. This device provides a facial mask that has two spaced layers of flexible sheet material and a suction device connected to the space between. It also has an expansive chamber at the rear of the mask. This device is too complicated and appears very uncomfortable to be worn while relaxing or sleeping. It might even create bruising, if the wearer does not know how to operate the air pump provided.

Kurimoto et al. (U.S. Pat. No. 5,623,733) discloses a moisture retaining face mask, attaching to the wearer's face with straps and fasteners. The mask includes a face covering portion that virtually covers the whole user's face. This device is primarily for moisturizing facial skin rather than to tone. It also seems very burdensome on the user since it covers the whole face, rather than partial covering.

While all of these masks and devices are in effect designed to improve facial features and retard the aging process, there is room for simple and economical improvements.

Disadvantages with prior art include: too complicated with crisscross bands in the back that only support the neck and bottom portion of the jaw; they do not provide much or any facial support; some can only be worn for a short period of time; adhesives that attach directly to the skin; they attach to the wearer, with no pressure and use magnets; most, if not all, are very uncomfortable to be worn while relaxing or sleeping; may create bruising, if the wearer does not know how to operate the air pump provided; and other devices seem very burdensome on the user since they cover the whole face, rather than partial covering.

Thus, there is a need for a facial toning and lifting device to provide a wearer with comfort while relaxing or even sleeping.

### SUMMARY OF THE INVENTION

Accordingly, the present invention provides a facial toning device made out of a one-piece stretchable material that contours around the bottom jaw line and around a wearer's head. There are openings for the chin and ears all reinforced for added comfort and shape retention. The device is held in place at the superior and posterior sides of the head by hook and loop fasteners. Adjustable bands are also provided allowing the wearer of the device to vary the amount of pressure exerted on the facial jaw line.

An optional eye attachment may be attached to the facial toning device, which the wearer may wear while sleeping or relaxing. The eye attachment includes a nose reinforcement to add shape and comfort to the nose area and eye gel pack holders that may be used to insert gel packs of the wearer's choice. The wearer may also use the optional eye attachment for adding darkness while sleeping. The optional eye attachment may be attached to the facial toning device by hook and loop fasteners.

While the invention has been described in conjunction with specific embodiments, it is evident that many alternatives, modifications and variations will be apparent to those skilled in the art in light of the foregoing description. Accordingly, the present invention attempts to embrace all such alternatives, modifications and variations that fall within the spirit and scope of the appended claims.

### BRIEF DESCRIPTION OF THE DRAWINGS

The invention will be illustrated and explained for a complete and thorough understanding, wherein:

FIG. 1 is a front perspective view of the facial device fitted on a human head;

FIG. 2 is a bottom perspective view of the facial device fitted on a human head;

FIG. 3 is a left planar view of the facial device, not fitted on a human head;

FIG. 4 is a right planar view of the facial device, not fitted on a human head;

FIG. 5 is a front planar view of the optional eye attachment, not fitted on a human head;

FIG. 6 is a back planar view of the optional eye attachment, not fitted on a human head;

FIG. 7 is a back perspective view of the facial device fitted on a human head; and

FIG. 8 is a planar view of the adjustable band assembly.

### DETAILED DESCRIPTION OF THE INVENTION

It is known in the art that applying pressure to muscles and skin is a technique that helps prevent and reduce sagging skin. Pressure therapy prevents sagging skin and helps tone the facial muscles. The present invention creates a youthful look by toning and firming the facial muscles through pressure in an easy to use, economical and comfortable device. It reduces lines and wrinkles and improves skin tone. These are especially desired following drastic weight-loss, such as in lipo-suction, where the person's skin is sagging. By applying pressure, which is also adjustable to the user's preference, the skin and muscles of the face will be significantly structured and toned. Another added use is that this invention can be comfortably worn during sleep. The invention performs all of the aforementioned remedies, but at the same time it is comfortable and very simple to operate.

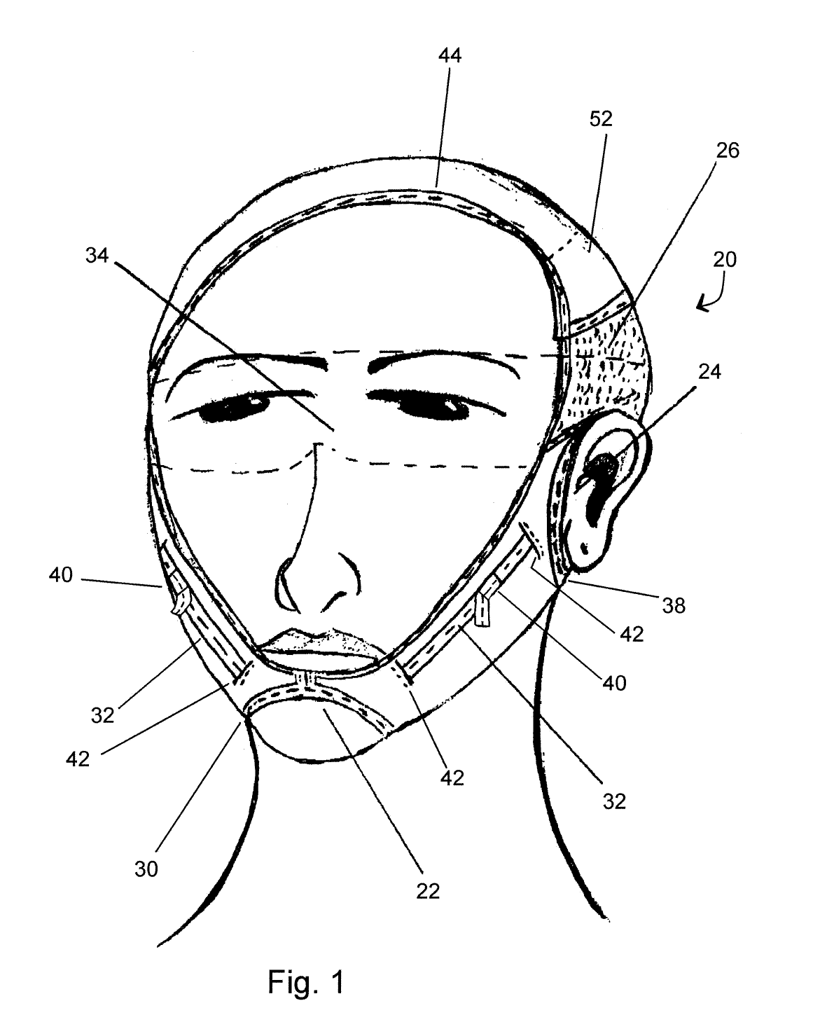

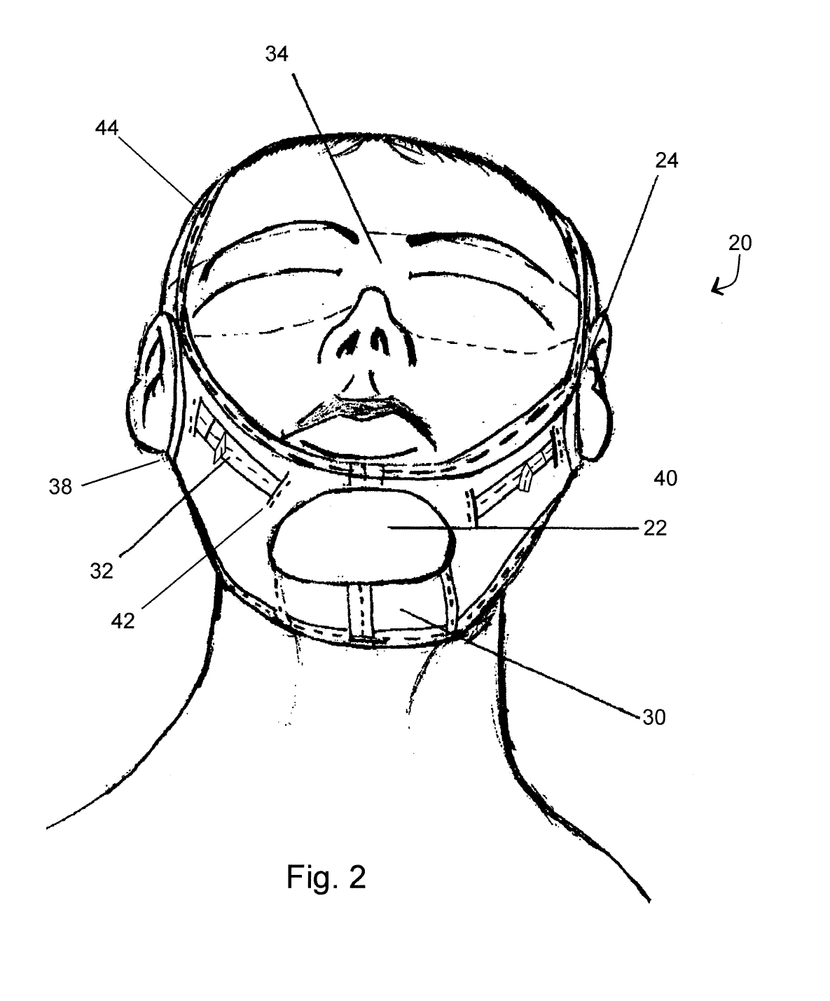

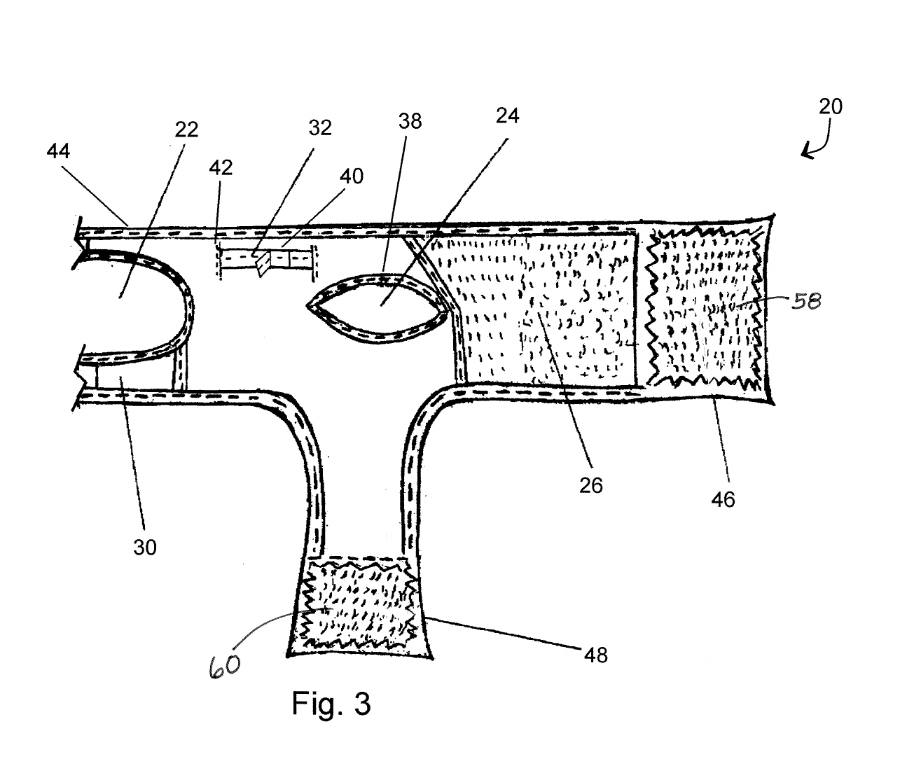

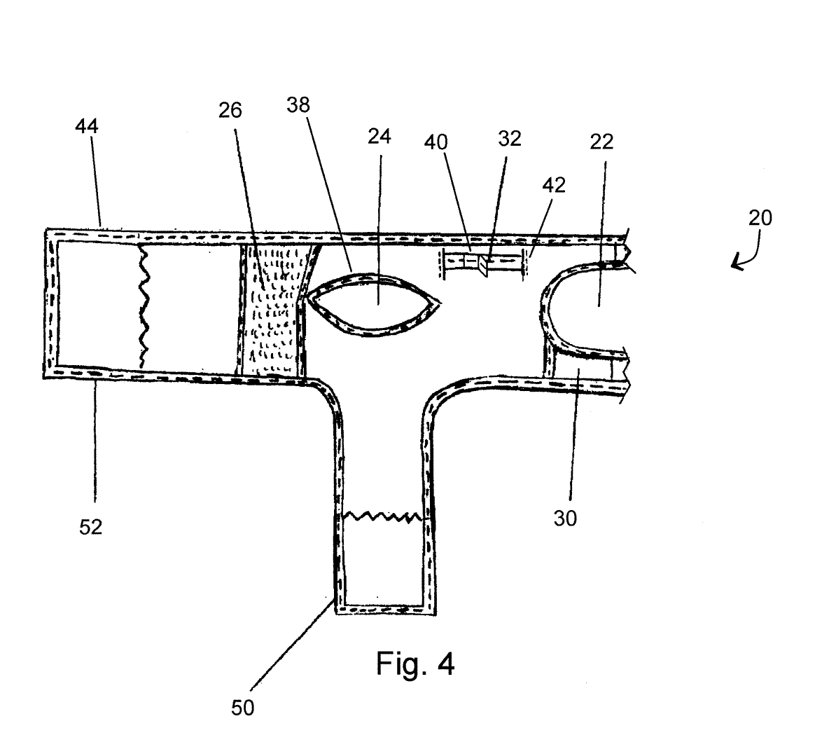

Referring now to the drawings, FIGS. 1-4 and 8 show an embodiment of the facial toning device of the present invention generally indicated at 20. FIGS. 3-4 show the facial device 20 in a planar view. In FIGS. 1-2 and 8, the facial device 20 is shown in use fitted on a human head. The facial device 20 may be made out of a suitable material known in the art that will allow stretching to apply a compressive force to a user's skin and muscles. Such a material might be those sold under the trade name LYCRA or SPANDEX, or any material that will allow the device to perform the function of applying pressure to the wearer's face. The facial device 20 may be any color one-piece stretchable material, having superior head attachment ends 46 and 52 and posterior head attachment ends 48 and 50, such as shown in FIG. 3, representing the left side of the facial device 20 and FIG. 4 representing the right side of the facial device 20. Along the edge of the facial device 20, there is a substantially strong, comforting facial reinforcement, for the device 20 to retain its form.

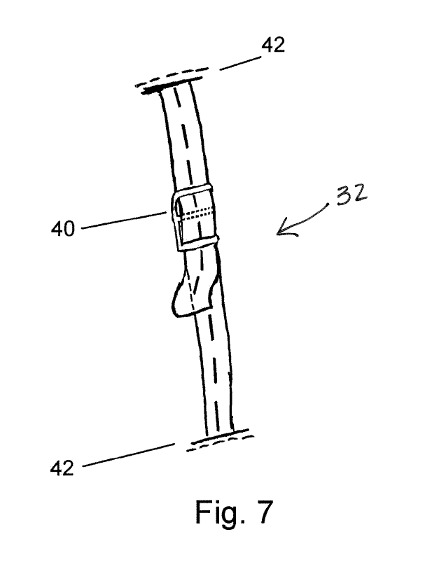

In FIGS. 1-4 and 8, there is shown an opening for the chin 22, which will allow the wearer to be substantially more comfortable while wearing the facial device 20 over their chin. The opening for the chin 22 has a chin reinforcement 30 surrounding it, which provides shape to the opening for the chin and gives it added support so that it may prevent the wearer from forming a double chin. Along the side of facial device 20 are two adjustable bands 32, which are better shown in FIG. 7. These adjustable bands 32 are attached to facial device 20 at the adjustable band ends 42, which may allow the wearer to vary the amount of pressure applied to the face. The wearer may vary the adjustable bands 32 by sliding on the adjustable slide clip 40, or any other similar device that can do the same task. Here, the adjustable slide clip 40 is the best mode. The tighter the wearer makes the bands, the more pressure will be applied to the wearer's face, and the contrary applies when the adjustable bands 32 are loosened. Above the adjustable straps 32 are ear openings 24, which allow the wearer's ears to protrude, the wearer to hear clearly, and be more comfortable. Ear reinforcements 38 add support to maintain shape and comfort to ear openings 24.

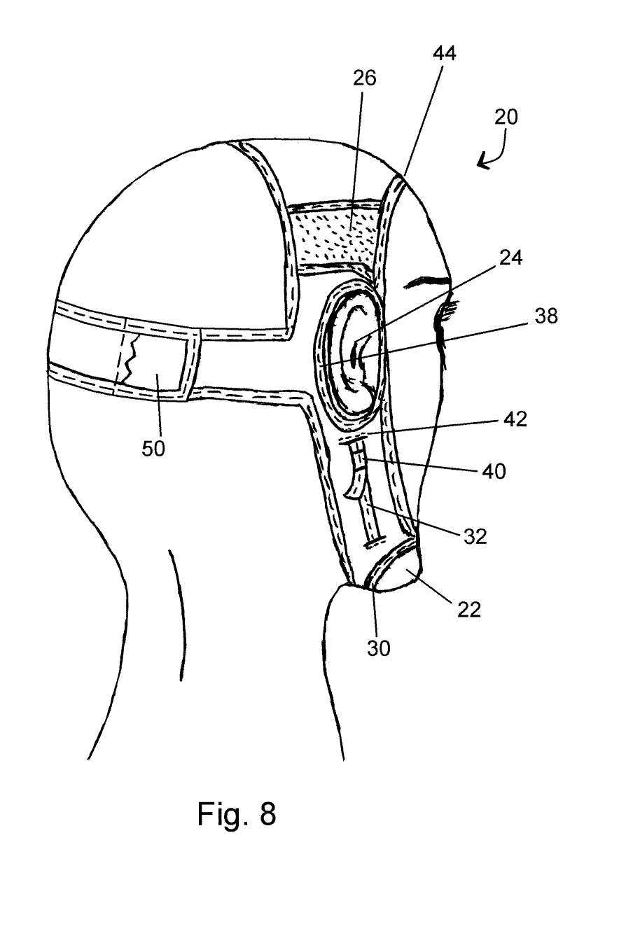

In FIGS. 3-4, facial device 20 is shown in planar view. Hook and loop fastener 58 (such as sold under the trade name VELCRO) is attached by any means known in the art to the superior head attachment ends 46 and 52 and fastened together on the superior side of the head, as shown in FIG. 1. Hook and loop fastener 60 is also attached by any means known in the art to the posterior head attachment ends 48 and 50, which meet on the posterior side of head and fastened, as shown in FIG. 8.

When facial device 20 is worn, as shown in FIGS. 1-2 and 8, the wearer first places their chin through the opening for the chin 22, then wraps the facial device 20 upward along the face and jaw line, while extending the wearer's ears through the ear openings 24. The wearer continues to stretch the mask along the head where hook and loop fastener 58 for the superior head attachment ends 46 and 52 attaches on the superior side of the head, as shown in FIG. 1. Next, the wearer joins the posterior head attachment ends 48 and 50 on the posterior side of the head, as shown in FIG. 8, where they are fastened together by hook and loop fastener 60. The superior and posterior head attachment ends 46, 52, 48 and 50 are designed so that they attach easily and can be detached easily from the wearer's head. When the wearer has fitted the facial device 20 on their head and attached the attachment ends 46 to 52 and 48 to 50 using hoop and loop fasteners 58 and 60, then the wearer can proceed to adjust the amount of pressure desired on their facial muscles by pulling on the adjustable bands 32.

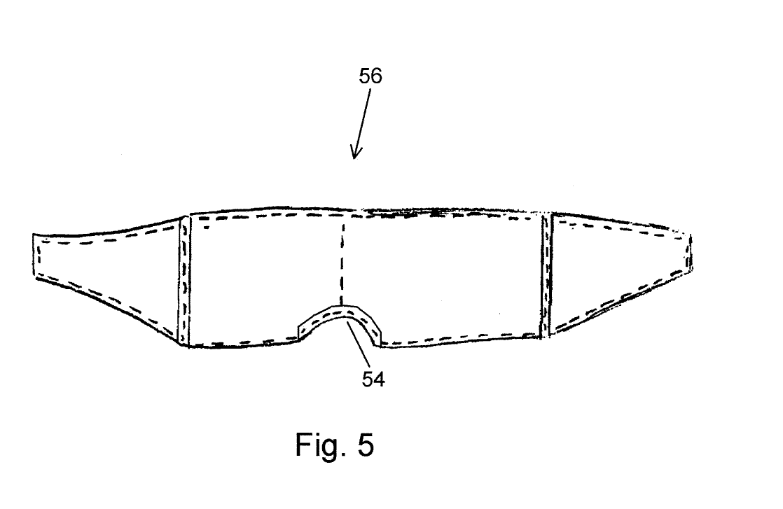

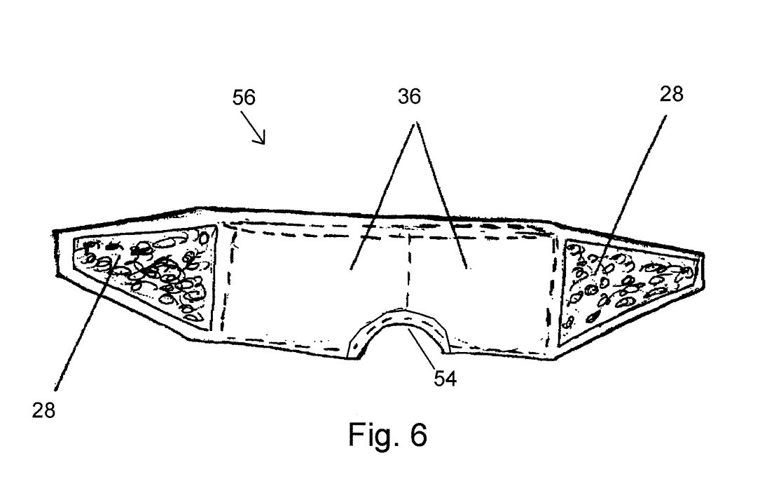

FIGS. 5-6 show an optional eye attachment 56, which can be worn while the wearer is wearing the facial device 20 while asleep or relaxing. Optional eye attachment 56 includes a front side (FIG. 5), a back side (FIG. 6), a nose reinforcement 54 to provide shape and comfort, and hook and loop fastener 28 on each end of the back side. Hook and loop fasteners 28 easily attach to the facial device 20 at hook and loop fasteners 26, in area 34 as shown in FIG. 1. On the optional eye attachment 56, in FIG. 6, eye gel pack holders 36 are shown, where the wearer may insert eye gel packs of their choice.

What is claimed is:

1. A facial toning device comprising:
an one-piece stretchable material, configured to apply a compressive force on the contours around the bottom jaw line and around a human head;
an annular ring defining an opening for the chin, having reinforcement;
a second and third annular ring configured to conform around an ear, said second and third annular ring having reinforcement;
a hook and loop fastener for a superior head attachment end, which will be contiguous with a hook and loop fastener for a second superior head attachment end, thereby allowing connection of the two ends of the facial toning device; and
adjustable bands, said adjustable bands having an adjustable slide clip that may attach to the facial toning device at adjustable band ends, wherein the wearer can adjust the amount of pressure exerted by the facial toning device.

2. The device of claim 1, further comprising a hook and loop fastener for a posterior head attachment end, configured to be contiguous with a hook and loop fastener for a second posterior head attachment end, thereby connecting the two ends of the facial toning device.

3. The device of claim 1, further comprising an eye attachment that may be contiguous with the facial toning device configured to cover the eyes of a user.

4. The device of claim 3, further comprising eye gel pack holders that may be used to insert gel packs of the wearer's choice.

5. The device of claim 3, wherein said eye attachment is configured to provide darkness to a user.

6. The device of claim 3, further comprising a nose reinforcement attached in the center of device.
